(19) United States
(12) Patent Application Publication
(10) Pub. No.: US 2023/0377694 A1
(43) Pub. Date: Nov. 23, 2023

(54) COMPOSITION SOLUBILITY IN AN AQUEOUS ENVIRONMENT COMPRISING A SURFACTANT MOLECULE DETERMINATION METHOD AND SYSTEM

(71) Applicant: Firmenich SA, Satigny (CH)

(72) Inventor: Wolfgang FIEBER, Satigny (CH)

(21) Appl. No.: 18/322,079

(22) Filed: May 23, 2023

(30) Foreign Application Priority Data
May 23, 2022 (EP) 22174764.5

(51) Int. Cl. G16C 20/30

(52) U.S. Cl. CPC G16C 20/30

(57) ABSTRACT

The composition solubility in an aqueous environment comprising a surfactant molecule determination method (100), comprises:
a step (105) of inputting at least one physical perfuming ingredient digital identifier, the resulting input being representative of a composition of the represented physical perfuming ingredients,
a step (110) of selecting at least one physical surfactant molecule digital identifier,
a step (115) of calculating a value representative of the total solubility of the corresponding physical perfuming ingredient in an aqueous environment, wherein the aqueous environment, at least one said selected physical surfactant, and the composition define a mixture,
a step (120) of computing, a value representative of the hydrophilic lipophilic difference of said mixture,
a step (125) of determining, a value representative of the composition solubility in the aqueous environment, and
a step (130) of providing at least one determined composition solubility.

### TECHNICAL FIELD OF THE INVENTION

The present invention relates to a composition solubility in an aqueous environment comprising a surfactant molecule determination method and a composition solubility in an aqueous environment comprising a surfactant molecule determination system. It applies, in particular, to the fields of flavor and fragrance design, consumer fragrances, consumer products for home care, body care, personal care, cosmetics and oral care.

### BACKGROUND OF THE INVENTION

Traditionally, fragrances (or compositions) are created and used in association with specific application chassis (or bases). In the event where a fragrance presents problems of solubility, the fragrance needs to be reworked until a soluble version is found. This process is often done on a trial-and-error basis, using general formulation knowledge and techniques used in that field.

For example, it is commonly known that the addition of solubilizers to the fragrance can improve solubility in the surfactant base. Fragrances can also be diluted with solvents generally used in perfumery. Nevertheless, the process is tedious and time consuming. Moreover, the addition of functional ingredients reduces the concentration of fragrance in the final application, and as a consequence the olfactive impact of that fragrance is reduced. Furthermore, large amounts of solubilizers have the disadvantage of reducing the sensorial impact of a fragrance after dilution of an aqueous surfactant-based application for personal care and home care. Therefore, an approach where the usage of functional ingredients can be rationalized and optimized, so that only the strictly necessary concentration is added, improves the olfactive performance of fragrances.

In the past very basic descriptors have been used to predict fragrance solubility such as the logarithm of the n-octanol-water partition coefficient (log P_{O/W}) for example. Such descriptors turned out to be insufficient.

Another approach is disclosed in the publication “How to Use the Normalized Hydrophilic-Lipophilic Deviation (HLDN) Concept for the Formulation of Equilibrated and Emulsified Surfactant-Oil-Water Systems for Cosmetics and Pharmaceutical Products”, by Jean-Louis Salager et al., published in Cosmetics 2020, 7, 57.

Such approach is limited to the hydrophilic-lipophilic deviation (HLD) model that describes the stability of oil-water-surfactant mixtures, i.e. microemulsions and emulsions.

However, the HLD model just allows a prediction of the tendency of how much oil/water can be solubilized in a microemulsion as a function of formulation parameters. Moreover, the HLD model works accurately only when both, oil and water, are present in high amounts, and most importantly in close to equal amounts. When one of the two phases is present in excess amounts, such as in the case for fragranced consumer products where within typical application conditions there can be as much as 95-98% of water and only 0.5-1% of oil, the predictions or approximations of fragrance oil solubilization are much less accurate or impossible.

As such, there exists no satisfying system allowing for the automatic determination of the solubilization capacity of a fragrance composition in an application base.

### SUMMARY OF THE INVENTION

The present invention is intended to remedy all or part of these disadvantages.

To this effect, according to a first aspect, the present invention aims at a composition solubility in an aqueous environment comprising a surfactant molecule determination method, comprising:
a step of inputting, upon a computer interface, at least one physical perfuming ingredient digital identifier, said physical perfuming ingredient digital identifier being representative of a physical perfuming ingredient, the resulting input being representative of a composition of the represented physical perfuming ingredients,
a step of selecting, upon a computer interface, at least one physical surfactant molecule digital identifier, said surfactant molecule digital identifier being representative of a physical surfactant molecule,
a step of calculating, by a computing system, for at least one input physical perfuming ingredient digital identifier, a value representative of the total solubility of the corresponding physical perfuming ingredient in an aqueous environment comprising micelles of at least one physical surfactant molecule corresponding to at least one selected physical surfactant molecule digital identifier, wherein the aqueous environment, at least one said selected physical surfactant, and the composition define a mixture,
a step of computing, by a computing system, for at least one input physical perfuming ingredient digital identifier in the composition and for at least one selected physical surfactant molecule digital identifier in the mixture, a value representative of the hydrophilic lipophilic difference of said mixture,
a step of determining, by a computing system, for at least one input physical perfuming ingredient digital identifier, a value representative of the composition solubility in the aqueous environment, as a function of a total solubility calculated and of a hydrophilic lipophilic difference computed, and
a step of providing, upon a computer interface, at least one determined composition solubility.

Such provisions allow for the prediction of the performance of a perfuming ingredient in an aqueous environment, relative to a determined application base. Such provisions further allow for the prediction of the performance of a perfuming composition in an aqueous environment, relative to a determined application base. zo Such provisions further allow for the increase in throughput in fragrance composition solubility screenings. Such provisions allow for the prediction of the solubility limit for a particular fragrance composition. Furthermore, such provisions allow for the dynamic formulation of fragrance compositions, providing the capacity for users to optimize the performance of their fragrance compositions.

In particular embodiments, the method object of the present invention further comprises a step of calculation, for the mixture, by a computing system, of a value representative of a maximum stability temperature, said temperature being used during the step of determining a value representative of the composition solubility in an aqueous environment comprising micelles of at least one selected surfactant molecule.

Such embodiments allow for the accurate prediction of the maximum stability temperature, above which the composition does not accurately deliver the level of performance.

In particular embodiments, the maximum stability temperature is calculated using the formula:

$$T = \frac{H L D - C c + k \cdot EACN - ln ( S ) + f ( A ) - t \cdot 25}{- t}$$

where:
HLD is the hydrophilic lipophilic difference of said mixture,
Cc is the characteristic curvature of the surfactant molecule,
EACN is the equivalent alkane carbon number of the perfuming ingredient,
S is the concentration of electrolyte in the mixture,
A is the concentration of alcohol in the mixture,
k, f, and t are constants.

In particular embodiments, the total solubility for a perfuming ingredient is calculated using the formula:

Sₜₒₜ^{F}=S_{W}^{F}·(1+P_{O/W}^{F}·AF·V̅_{S}·c_{S}/(1−V̅_{S}·c_{S}))

where:
S_{W}^{F} is the perfuming ingredient solubility in water,
c_{S} is the concentration of micellized surfactant in the mixture,
V̅_{S} is the molecular volume of the surfactant molecule,
P_{O/W}^{F} is the n-octanol-water partition coefficient of the perfuming ingredient and
AF is the affinity factor of the surfactant molecule.

In particular embodiments, the hydrophilic lipophilic difference for a perfuming ingredient is calculated using the formula:

HLD=ln(S)−k·EACN−f(A)+Cc−t·(ΔT)

where:
S is the concentration of electrolyte in the mixture,
EACN is the equivalent alkane carbon number of the perfuming ingredient,
A is the concentration of alcohol in the mixture,
ΔT is the temperature difference to a reference temperature of the mixture,
Cc is the characteristic curvature of the surfactant molecule,
k, f, and t are constants.

In particular embodiments, the method object of the present invention further comprises a step of determining, by a computing system, an adjusted quantity of at least one input physical perfuming ingredient represented by at least one input physical perfuming ingredient digital identifier as a function of the determined composition solubility in an aqueous environment to reach a target hydrophilic lipophilic difference, the step of providing being configured to provide the determined adjusted concentration.

Such embodiments allow for the dynamic adjustment of a composition by reducing the quantity of a perfuming ingredient that negatively impacts the solubility performance of the composition.

In particular embodiments, the method object of the present invention further comprises a step of determining, by a computing system, at least one additional physical solubilizer digital identifier, representative of a physical solubilizer, to be input in the composition as a function of the determined composition solubility in the aqueous environment to reach a target hydrophilic lipophilic difference, the step of providing being configured to provide the determined additional solubilizer digital identifier.

Such embodiments allow for the dynamic adjustment of a composition by adding molecules that positively impact the solubility performance of the composition.

In particular embodiments, the method object of the present invention further comprises a step of determining, by a computing system, at least one additional physical solvent digital identifier, representative of a physical solvent, to be input in the composition as a function of the determined composition solubility in the aqueous environment to reach a target hydrophilic lipophilic difference, the step of providing being configured to provide the determined additional solvent digital identifier.

Such embodiments allow for the dynamic adjustment of a composition by adding molecules that positively impact the solubility performance of the composition.

In particular embodiments, the method object of the present invention further comprises a step of determining, by a computing system, at least one physical perfuming ingredient digital identifier to be removed as a function of the determined composition solubility in an aqueous environment to reach a target hydrophilic lipophilic difference, the step of providing being configured to provide the determined corresponding physical perfuming ingredient digital identifier.

Such embodiments allow for the dynamic adjustment of a composition by removing perfuming ingredient that negatively impacts the solubility performance of the composition.

In particular embodiments, the method object of the present invention further comprises:
a step of comparing, by a computing system, the determined composition solubility in an aqueous environment to at least one threshold value and
a step of determination, by a computing system, of at least one physical cause digital identifier for the result of the step of comparing,
   the step of providing being configured to provide each said physical cause digital identifier.

Such embodiments allow for providing, to a fragrance designer, reasons for the lack of performance of a composition to avoid empirical trial and error approaches.

In particular embodiments, the method object of the present invention further comprises a step of assembling the composition.

Such embodiments allow for the materialization of the digitalized composition.

In particular embodiments, the method object of the present invention further comprises a step of constructing at least one database associating:
physical perfuming ingredient digital identifiers to at least one physical perfuming ingredient parameter value, and
physical surfactant molecule digital identifiers to at least one physical surfactant molecule parameter value,
   wherein at least one said physical perfuming ingredient parameter value and at least one physical surfactant molecule parameter value are used during the step of calculating and/or the step of computing.

According to a second aspect, the present invention aims at a composition solubility in an aqueous environment comprising a surfactant molecule determination system, comprising:
means of inputting, upon a computer interface, at least one physical perfuming ingredient digital identifier, said physical perfuming ingredient digital identifier being representative of a physical perfuming ingredient, the resulting input being representative of a composition of the represented physical perfuming ingredients,
means of selecting, upon a computer interface, at least one physical surfactant molecule digital identifier, said surfactant molecule digital identifier being representative of a physical surfactant molecule,
means of calculating, by a computing system, for at least one input physical perfuming ingredient digital identifier, a value representative of the total solubility of the corresponding physical perfuming ingredient in an aqueous environment comprising micelles of at least one physical surfactant molecule corresponding to at least one selected physical surfactant molecule digital identifier, wherein the aqueous environment, at least one said selected physical surfactant, and the composition define a mixture,
means of computing, by a computing system, for at least one input physical perfuming ingredient digital identifier in the composition and for at least one selected physical surfactant molecule digital identifier in the mixture, a value representative of the hydrophilic lipophilic difference of said mixture,
means of determining, by a computing system, for at least one input physical perfuming ingredient digital identifier, a value representative of the composition solubility in the aqueous environment, as a function of a total solubility calculated and of a hydrophilic lipophilic difference computed, and
means of providing, upon a computer interface, at least one determined composition solubility.

The system object of the present invention presents the same advantages as the method object of the present invention.

### BRIEF DESCRIPTION OF THE DRAWINGS

Other advantages, purposes and particular characteristics of the invention shall be apparent from the following non-exhaustive description of at least one particular method or system which is the object of this invention, in relation to the drawings annexed hereto, in which:

FIG. 1 represents, schematically, a first particular succession of steps of the method subject of the present invention,

FIG. 2 represents, schematically, a second particular succession of steps of the method subject of the present invention,

FIG. 3 represents, schematically, a particular embodiment of the system subject of the present invention,

FIG. 4 represents, schematically, a first two-dimensional graph representative of the performance of a composition in terms of solubility,

FIG. 5 represents, schematically, a second two-dimensional graph representative of the performance of a composition in terms of solubility,

FIG. 6 represents, schematically, a two-dimensional graph representative of the performance of the prediction of the model object of the present invention, and

FIG. 7 represents, schematically, a computing device capable of implementing a method object of the present invention.

### DETAILED DESCRIPTION OF THE INVENTION

This description is not exhaustive, as each feature of one embodiment may be combined with any other feature of any other embodiment in an advantageous manner.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood as inclusive.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should be noted at this point that the figures are not to scale.

As used herein, the terms “perfuming ingredient” it is meant here a compound, which is used in a perfuming preparation or a composition to impart a hedonic effect, i.e., used for the primary purpose of conferring or modulating an odor. In other words, such an ingredient, to be considered as being a perfuming one, must be recognized by a person skilled in the art as being able to impart or modify in a positive or pleasant way the odor of a composition, and not just as having an odor. The perfuming ingredient may impart an additional benefit beyond that of modifying or imparting an odor, such as long-lasting, blooming, malodor counteraction, antimicrobial effect, antiviral effect, microbial stability, or pest control.

The nature and type of the perfuming co-ingredients present in the base do not warrant a more detailed description here, which in any case would not be exhaustive, the skilled person being able to select them on the basis of his general knowledge and according to the intended use or application and the desired organoleptic effect. In general terms, these perfuming ingredients belong to chemical classes as varied as alcohols, lactones, aldehydes, ketones, esters, ethers, acetates, nitriles, terpenoids, nitrogenous or sulfurous heterocyclic compounds and essential oils, and said perfuming ingredients can be of natural or synthetic origin. Perfuming Ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

The terms “composition” or formula designates a liquid, solid and/or gaseous assembly of at least one perfuming ingredient.

As used herein, a “fragrance” refers to the olfactory perception resulting from the sum of odorant receptor(s) activation, enhancement, and inhibition (when present) by at least one perfuming ingredient. Accordingly, by way of illustration and by no means intending to limit the scope of the present disclosure, a “fragrance” results from the olfactory perception arising from the sum of a first perfuming ingredient that activates an OR associated with a coconut tonality, a second perfuming ingredient that activates an OR associated with a celery tonality, and a third perfuming ingredient that inhibits an OR associated with a hay tonality.

As used herein, the terms “means of inputting” is, for example, a keyboard, mouse and/or touchscreen adapted to interact with a computing system in such a way to collect user input. In variants, the means of inputting are logical in nature, such as a network port of a computing system configured to receive an input command transmitted electronically. Such an input means may be associated to a GUI (Graphic User Interface) shown to a user or an API (Application programming interface). In other variants, the means of inputting may be a sensor configured to measure a specified physical parameter relevant for the intended use case.

As used herein, the terms “computing system” or “computer system” designate any electronic calculation device, whether unitary or distributed, capable of receiving numerical inputs and providing numerical outputs by and to any sort of interface, digital and/or analog. Typically, a computing system designates either a computer executing a software having access to data storage or a client-server architecture wherein the data and/or calculation is performed at the server side while the client side acts as an interface.

As used herein, the terms “digital identifier” refer to any computerized representation identifier, such as one used in a computer database, representing a physical object, such as a physical perfuming ingredient. A digital representation identifier may refer to a label representative of the name, chemical structure or internal reference of the physical perfuming ingredient. Such a representation is bijective, meaning that one physical perfuming ingredient corresponds to one physical perfuming ingredient digital representation identifier and vice versa.

In the present description, the term ‘materialized’ or ‘physical’ or ‘real’ is intended as existing outside of the digital environment of the present invention. ‘Materialized’ may mean, for example, readily found in nature or synthesized in a laboratory or chemical plant. In any event, a materialized composition presents a tangible reality. The terms ‘to be compounded’ or ‘compounding’ refer to the act of materialization of a composition, whether via extraction and assembly of ingredients or via synthetization and assembly of ingredients.

The present invention can be considered, in particular embodiments, as a predictive model that can be used as a help for faster screening of fragrance solubility and giving guidelines for reformulation. Such a model is a holistic model that considers both surfactant base and fragrance. Important formulation variables such as fragrance composition and concentration, surfactant concentration and surfactant type, and the effect of functional additives such as solvents and solubilizers can be included.

It should be noted that in aqueous surfactant systems the surfactant molecules are typically organized in the form of micelles above the critical micellar concentration. Perfuming ingredients can be solubilized in the hydrophobic interior of the micelles, yielding a transparent liquid phase without phase separation. Nevertheless, the quantity of a composition that a micelle can solubilize is limited and depends on the concentration of micellized surfactant molecules. As a consequence, when the added amount of a perfuming composition exceeds the solubilization capacity a phase separation takes place that leads to instability and turbidity, which is undesirable in a consumer product.

In the present description, the term “mixture” refers to the combination of at least a fragrance composition, a surfactant and water. Optionally, the mixture can be extended to also refer to additionally include electrolytes, solvents, alcohol, solubilizers and other additives such as colorants, preservatives, antimicrobial agents, opacifiers, emollients, humectants, antioxidants, free radical scavengers, POV remediation agents, cooling agents, vitamins, insect repellents, fixatives, cosmetic benefit agents, chelators, functional polymers, and pH adjusters.

Such an invention particularly applies to perfumed consumer products.

Non-limiting examples of suitable perfumed consumer products include a perfume, such as a fine perfume, a splash or eau de parfum, a cologne or a shave or after-shave lotion; a fabric care product, such as a liquid or solid detergent optionally in the form of a pod or tablet, a fabric softener, a liquid or solid scent booster, a dryer sheet, a fabric refresher, an ironing water, a paper, a bleach, a carpet cleaner, a curtain-care product; a body-care product, such as a hair care product (e.g. a shampoo, a leave-on or rinse-off hair conditioner, a coloring preparation or a hair spray, a color-care product, a hair shaping product, a dental care product), a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.); or a home care product, such as a mold remover, a furnisher care product, a wipe, a dish detergent or a hard-surface (e.g. a floor, bath, sanitary or a window-cleaning) detergent; a leather care product; a car care product, such as a polish, a wax or a plastic cleaner.

According to any embodiments of the invention, the perfumed consumer product of the invention are characterized by a pH of 1 or more. Particularly, the perfumed consumer product of the invention has a pH comprised between 1 and 12, or between 1 and 8. Even more particularly, the perfumed consumer product of the invention has a pH comprised between 1 and 6.

According to a particular embodiment, the invention's perfumed consumer product is in the form of a personal care, a home care or fabric care consumer product comprising ingredients that are common in personal, home or fabric care consumer products, in particular shower gels, shampoos, soaps, fabric detergents or softeners and all-purpose cleaners. The main functional constituents of perfumed consumer products are surfactants and/or softener components capable of cleaning and/or softening fabrics and/or textiles of varied nature, such as clothes, curtain fabrics, carpets and furniture fabrics, etc, or other home surfaces, skin or hair, and typically used in a large amount of water or water-based solvents. These are therefore formulations wherein the amount of water is typically comprised between 50 and 99% by weight of the perfumed consumer product with the exception of soaps or solid detergents wherein the amount of water is at most 20%.

A more detailed description of such fabric cleaning and/or softening formulations is not warranted here, many descriptions of current liquid formulations can be found in the cleaner/fabric softener's patent and other pertinent literature, such as for example the textbook of Louis Ho Tan Tai, “Détergents et Produits de Soins Corporels, Chapters 1 to 7 in particular, Dunod, Paris, 1999, or any other similar and/or more recent textbooks pertaining to the art of liquid softener and all-purpose cleaners formulations. A patent publication, WO 2010/105873, is also cited by way of example, in as much as it describes typical current ingredients, other than perfumes, of such liquid products, particularly in pages 9 to 21. Of course, many other examples of liquid cleaner and/or fabric softener formulations can be found in the literature. Any such liquid formulations, namely liquid fabric cleaners or conditioners and/or all-purpose cleaners, can be used in the here-described compositions.

According to a particular embodiment of the invention, the invention's perfumed consumer product is a liquid fabric softener comprising a fabric softener active base in amount comprised between 85 and 100% by weight, based on the total weight of the perfumed consumer product. The main constituent of the fabric softener active base is water or water-based solvents. The fabric softener active base may comprise dialkyl quaternary ammonium salts, dialkyl ester quaternary ammonium salts, Hamburg esterquat, triethanolamine quat, silicones and mixtures thereof. Optionally, the fabric softener active base of the composition may further comprise a viscosity modifier in an amount comprised between 0.05 and 1% by weight, based on the total weight of the liquid base; preferably chosen in the group consisting of calcium chloride.

According to a particular embodiment of the invention, the invention's perfumed consumer product is an all-purpose cleaner comprising an all-purpose cleaner active base in amount comprised between 85 and 100% by weight, based on the total weight of the perfumed consumer product. The main constituent of the all-purpose cleaner active base is water or water-based solvents The all-purpose active base may comprise linear alkylbenzene sulfonates (LAS) in an amount comprised between 1 and 2%, nonionic surfactant in an amount comprised between 2 and 4% and acid such as citric acid in an amount comprised between 0.1 and 0.5%.

According to a particular embodiment of the invention, the invention's perfumed consumer product is a liquid detergent comprising a liquid detergent active base in amount comprised between 85 and 100% by weight, based on the total weight of the perfumed consumer product. The main constituent of the liquid detergent active base is water or water-based solvents. The liquid detergent active base may comprise anionic surfactant such as alkylbenzenesulfonate (ABS), linear alkylbenzene sulfonates (LAS), secondary alkyl sulfonate (SAS), primary alcohol sulfate (PAS), lauryl ether sulfate (LES), sodium lauryl ether sulfate (SLES), methyl ester sulfonate (MES); nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxides, alkyl polyglucosides, alkyl polyglucosamides; or mixtures thereof.

According to a particular embodiment of the invention, the invention's perfumed consumer product is a solid detergent comprising a solid detergent active base in amount comprised between 85 and 100% by weight, based on the total weight of the perfumed consumer product. The solid detergent active base may comprise at least one surfactant chosen in the group consisting of anionic, nonionic, cationic, zwiterionic surfactant and mixtures thereof. The surfactant in the solid detergent active base is preferably chosen in the group consisting of linear alkene benzene sulfonate (LABS), sodium laureth sulphate, sodium lauryl ether sulfate (SLES), sodium lauryl sulfate (SLS), alpha olefin sulfonate (AOS), methyl ester sulfonates (MES), alkyl polyglyucosides (APG), primary alcohol ethoxylates and in particular lauryl alcohol ethoxylates (LAE), primary alcohol sulphonates (PAS), soap and mixtures thereof. The soild detergent active base may comprise a further component, commonly used in powder detergent consumer product, selected from the group consisting of bleaching agents such as TAED (tetraacetylethylenediamine); buffering agent; builders such as zeolites, sodium carbonate or mixture thereof; soil release or soil suspension polymers; granulated enzyme particles such as cellulase, lipase, protease, mannanase, pectinase or mixtures thereof; corrosion inhibitor; antifoaming; sud suppressing agents; dyes; fillers such as sodium silicate, sodium sulfate or mixture thereof; source of hydrogen peroxide such as sodium percarbonate or sodium perborate; and mixtures thereof.

According to a particular embodiment of the invention, the invention's perfumed consumer product is shampoo or a shower gel comprising a shampoo or shower gel active base in amount comprised between 85 and 100% by weight, based on the total weight of the perfumed consumer product. The main constituent of the shampoo or a shower gel active base is water or water-based solvents The shampoo shower gel active base may comprise sodium alkylether sulfate, ammonium alkylether sulfates, alkylamphoacetate, cocamidopropyl betaine, cocamide MEA, alkylglucosides and aminoacid based surfactants.

According to a particular embodiment of the invention, the invention's perfumed consumer product is a soap bar comprising a soap active base in amount comprised between 85 and 100% by weight, based on the total weight of the perfumed consumer product. The soap bar active base may comprise salt of a weak acid, typically, a salt of weak acid, which may be a fatty acid and strong base like sodium hydroxide.

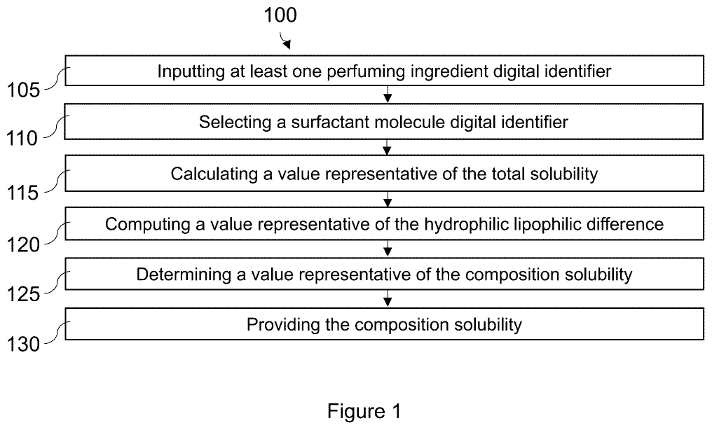

FIG. 1 shows a particular succession of steps of the method 100 object of the present invention. This composition solubility in an aqueous environment comprising a surfactant molecule determination method 100 comprises:
a step 105 of inputting, upon a computer interface, at least one physical perfuming ingredient digital identifier, said physical perfuming ingredient digital identifier being representative of a physical perfuming ingredient, the resulting input being representative of a composition of the represented physical perfuming ingredients,
a step 110 of selecting, upon a computer interface, at least one physical surfactant molecule digital identifier, said surfactant molecule digital identifier being representative of a physical surfactant molecule,
a step 115 of calculating, by a computing system, for at least one input physical perfuming ingredient digital identifier, a value representative of the total solubility of the corresponding physical perfuming ingredient in an aqueous environment comprising micelles of at least one physical surfactant molecule corresponding to at least one selected physical surfactant molecule digital identifier, wherein the aqueous environment, at least one said selected physical surfactant, and the composition define a mixture,
a step 120 of computing, by a computing system, for at least one input physical perfuming ingredient digital identifier in the composition and for at least one selected physical surfactant molecule digital identifier in the mixture, a value representative of the hydrophilic lipophilic difference of said mixture,
a step 125 of determining, by a computing system, for at least one input physical perfuming ingredient digital identifier, a value representative of the composition solubility in the aqueous environment, as a function of a total solubility calculated and of a hydrophilic lipophilic difference computed, and
a step 130 of providing, upon a computer interface, at least one determined composition solubility.

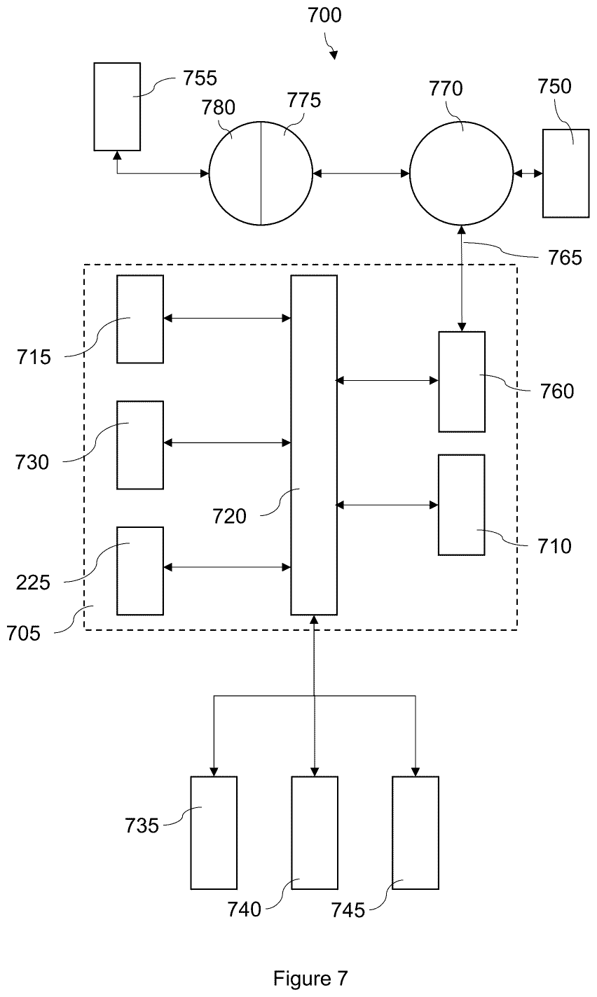

The step of inputting 105 is performed, for example, by using any means of inputting associated with a computing system, such as shown in FIG. 7. For example, during this step of inputting 105, a user may select, upon a GUI, at least one physical perfuming ingredient digital identifier among a list of physical perfuming ingredient digital identifiers. Such an identifier may be, for example, the name of the perfuming ingredient or a code or reference representative of such a perfuming ingredient.

In more advanced embodiments, the step of inputting 105 comprises a step of defining operational parameters of the input physical perfuming ingredient digital identifiers. Such operational parameters correspond to, for example, a quantity (in absolute or relative terms) of said input perfuming ingredient.

In more advanced embodiments, the step of inputting 105 comprises a step of inputting additive digital identifiers, representing materialized solvents or solubilizers, to the composition.

This step of inputting 105 may be performed in one or more sub-steps performed independently during the execution of the method 100 object of the present invention.

At the end of this step of inputting 105, an initial composition is obtained, said composition or formula being representative of a real composition or formula to be materialized.

The step of selecting 110 is performed, for example, by using any means of inputting associated with a computing system, such as shown in FIG. 7. For example, during this step of selecting 110, a user may select, upon a GUI, at least one surfactant molecule digital identifier among a list of surfactant molecule digital identifiers. Such an identifier may be, for example, the name of the surfactant molecule or a code or reference representative of such a surfactant molecule.

In more advanced embodiments, the step of selecting 110 comprises a step of defining operational parameters of the input surfactant molecule digital identifier. Such operational parameters correspond to, for example, a quantity (in absolute or relative terms) of said input surfactant molecule.

The step of calculating 115 is performed, for example, by a computer program executed by a computing system, such as shown in FIG. 7. There are different applicable equations and methods to compute the total solubility of perfuming ingredients represented by digital identifiers.

In one such method, the following equation is used:

Sₜₒₜ^{F}=S_{W}^{F}·(1+P_{O/W}^{F}·AF·V̅_{S}·c_{S}/(1−V̅_{S}·c_{S}))

where:
S_{W}^{F} is the perfuming ingredient solubility in water, such a value being either taken from experimental values, predicted values with different algorithms (e.g., calculated with the software EPI Suite), or estimated based on a model using log P, such as, for example: log S_{W}=−log P_{O/W}+0.8 (Encyclopedia of Pharmaceutical Technology, Vol. 18, p. 161),
c_{S} is the concentration of micellized surfactant in the mixture,
V̅_{S} is the molecular volume of the surfactant molecule,
P_{O/W}^{F} is the n-octanol-water partition coefficient of the perfuming ingredient and
AF is the affinity factor of the surfactant molecule, this affinity factor determines the partition coefficient of the perfuming ingredient between micelles and water—examples of values of such parameters may be obtained, for example, using the methodology disclosed in Fieber et. al. “Competition between surfactants and apolar fragrances in surfactant micelles.” Colloids and Surfaces A 539 (2018), 310-318.

Once, the maximum solubility is calculated, it is possible to determine the insoluble part of the perfuming ingredients with the following formula:

ϕ_{insoluble}^{F}=c^{F}−Sₜₒₜ^{F}

Where:
ϕ_{insoluble}^{F} represents the concentration of the insoluble fraction of a perfuming ingredient,
c^{F} represents the concentration of the perfuming ingredient in the mixture and
Sₜₒₜ^{F} represents the total solubility of the perfuming ingredient in the mixture.

Such an equation allows for the calculation of the total insoluble fraction, obtained with the following formula for example:

$${Φ}_{i n s o l u b l e}^{t o t} = {\sum }_{i = 1}^{n} {ϕ}_{i n s o l u b l e , n}^{F}$$

This insoluble fraction is then representative of the performance of the composition given the particular surfactant selected. The lower the insoluble fraction, the more performing is the composition. Preferably, the total solubility of the perfuming ingredient is equal or greater than the concentration of the perfuming ingredient in the mixture, in which case the total insoluble fraction is zero.

The step 120 of computing a hydrophilic lipophilic difference is performed, for example, by a computer program executed by a computing system. There are different applicable equations and methods to compute the hydrophilic lipophilic difference of a composition represented by digital identifiers.

According to one such approach, the following equation may be used:

HLD=ln(S)−k·EACN−f(A)+Cc−t·(ΔT)

where:
S is the concentration of electrolyte in the mixture,
EACN is the equivalent alkane carbon number of the perfuming ingredient,
A is the concentration of alcohol in the mixture,
ΔT is the temperature difference to a reference temperature of the mixture (typically 25° C.),
Cc is the characteristic curvature of the surfactant molecule,
k, f, and t are constants.

The HLD (hydrophilic lipophilic difference) concept (Salager et al., Partitioning of Ethoxylated Octylphenol Surfactants in Microemulsion-Oil-Water Systems: Influence of Temperature and Relation between Partitioning Coefficient and Physicochemical Formulation. Langmuir 16 (2000), 5534) is an attempt to capture the balance between water and oil phases in a microemulsion by relating both surfactant and oil polarity. In addition, other formulation parameters such as temperature, salt and solvents are considered, which makes it much more powerful than simple molecular descriptors such as HLB or log P. This semi-empirical concept has been found very useful in the formulation of microemulsions because all the formulation parameters are considered. A system containing surfactant with equal volumes of oil and water is in equilibrium when the net surfactant-oil interactions are equal to the net surfactant-water interactions (R=1). In that case HLD equals to zero. This is defined as the optimum formulation.

EACN (equivalent alkane carbon number) expresses the polarity of an organic compound and compatibility with the surfactant, respectively. EACN can be determined experimentally. The use of EACN as a classification tool and a method to determine EACN was published previously (Tchakalova and Fieber “Classification of fragrances and fragrance mixtures based on interfacial solubilization.” J. Surfact. Deterg 15 (2012), 167-177).

Measurement of equivalent alkane carbon number (EACN) of a perfume ingredient in model system:

Different fragrance compositions are added to the model microemulsion system shown in Table 1, in which the nonionic pentaethylene glycol mono n-decyl ether (C10E5) is used as surfactant. Phase transition temperatures of the system from Winsor I to Winsor III, and from Winsor III to Winsor II are determined by stepwise heating the microemulsions in a water bath. Alkanes with different alkane carbon numbers (octane, decane, dodecane, tetradecane and hexadecane) are used to establish a linear calibration curve between EACN and the PIT (phase inversion temperature), which is the average of the transitions from Winsor I to Winsor III, and from Winsor III to Winsor II, respectively. EACNₘᵢₓ is then calculated from the measured PIT for systems containing different fragrance compositions based on the previously established linear relationship. The EACN of the pure fragrance composition is calculated based on the molar ratio of the tested PRMs in the oil phase using the following equation:

EACNₘᵢₓ=EACN×n+EACN_{ref}×n_{ref}

wherein n and n_{ref} are the molar fractions of tested perfume ingredient and the reference ingredient (isopropyl myristate) in the oil phase, respectively.

TABLE 1
Compositions for EACN measurement

| Model system | Conc. (% wt) |
|---|---|
| C10E5 | 10 |
| H₂O | 45 |
| Isopropyl | 42 |
| myristate | |
| Fragrance | 3 |
| composition | |

The characteristic curvature, which describes the surfactant polarity and its ability to form microemulsions can be determined experimentally. Such a value may be input as a part of the method 100 object of the present invention.

Measurement of characteristic curvature Cc:

The characteristic curvature of a surfactant molecule or a mixture of surfactant molecules is determined from salt formulation scans carried out with a reference oil, hexadecane, and a reference surfactant, Tergitol 15-S-5. The test surfactant is mixed with Tergitol 15-S-5 in a ratio 10:90. To 0.1 g of this mixture are added 1 mL of hexadecane and 1 mL of an aqueous NaCl solution. A series of samples are prepared with increasing concentrations of NaCl and placed in transparent vials. The mixtures are shaken vigorously and left at 25° C. for 24 h. The NaCl concentration is chosen in such a range that 2-phase to 3-phase and 3-phase to 2-phase transitions, respectively, with increasing concentration are visible. All samples are then shaken simultaneously. The sample for which the three phases separate the fastest is considered as the composition representing optimal salinity, S*ₘᵢₓ (Zarate-Munoz et al. J. Surf. Deterg. 19 (2016), 249-263).

The same procedure was carried out for pure Tergitol 15-S-5, which was used as reference surfactant. Cc of the test surfactants were then determined according to the following equation:

$$C {c}_{test} = C {c}_{ref} - ( \frac{{S}_{m i x}^{*} - {S}_{ref}^{*}}{{f}_{test}} ) \cdot b$$

Where S* is the optimal salinity determined for reference surfactant and the mixtures, respectively, fₜₑₛₜ is the molar fraction of the test surfactant in the mixture, and b was set to 0.13.

The important point of this approach is that the effect of solubilizers can be considered. Solubilizers are typically polar surfactants with a known Cc. In the present method 100, it is possible to calculate the Cc of the mixture with the surfactant base, which shifts the HLD to higher or to lower values. Such a Cc may be calculated using the following equation:

Ccₜₒₜₐₗ=f_{solubilizer}·CC_{solubilizer}+f_{basesurfactant}·CC_{basesurfactant}

where:
f_{solubilizer} represents the molar fraction of added solubilizer and
f_{base surfactant} represents the molar fraction of surfactant molecules in the base.

Such values can be calculated on the basis of the ratio between the moles of added solubilizer and the total moles of surfactant and solubilizer.

The above parameters may be retrieved from at least one database associating:
physical perfuming ingredient digital identifiers to at least one physical ingredient parameter value, and
physical surfactant molecule digital identifiers to at least one physical surfactant parameter value.

In particular embodiments, the method 200 object of the present invention comprises a step 206 of constructing at least one database associating:
physical perfuming ingredient digital identifiers to at least one physical perfuming ingredient parameter value, and
physical surfactant molecule digital identifiers to at least one physical surfactant molecule parameter value,
   wherein at least one said physical perfuming ingredient parameter value and at least one physical surfactant molecule parameter value are used during the step 115 of calculating and/or the step 120 of computing.

Such a step 206 of constructing may be performed by measuring a parameter value for at least one physical perfuming ingredient parameter value and storing the measured value, in a database, in relation to the physical perfuming ingredient digital identifier.

Such a step 206 of constructing may be performed by measuring a parameter value for at least one physical surfactant molecule parameter value and storing the measured value, in a database, in relation to the physical surfactant molecule digital identifier.

The step of determining 125 is performed, for example, by a computer software executed by a computing system, such as shown in FIG. 7. During this step 125 of determining, a value representative of the composition solubility in an aqueous environment comprising a surfactant molecule is determined as a function of a total solubility calculated and of a hydrophilic lipophilic difference computed.

This value may be a single value, obtained via a mathematical combination of the two values, or coordinates in a system in which one axis represents the total solubility calculated (or subsequent insoluble fraction determined) and one axis represents the hydrophilic lipophilic difference.

The step of providing 130 is performed, for example, by any computer interface suited for the particular use case. As such, this computer interface may be a GUI or an API, for example.

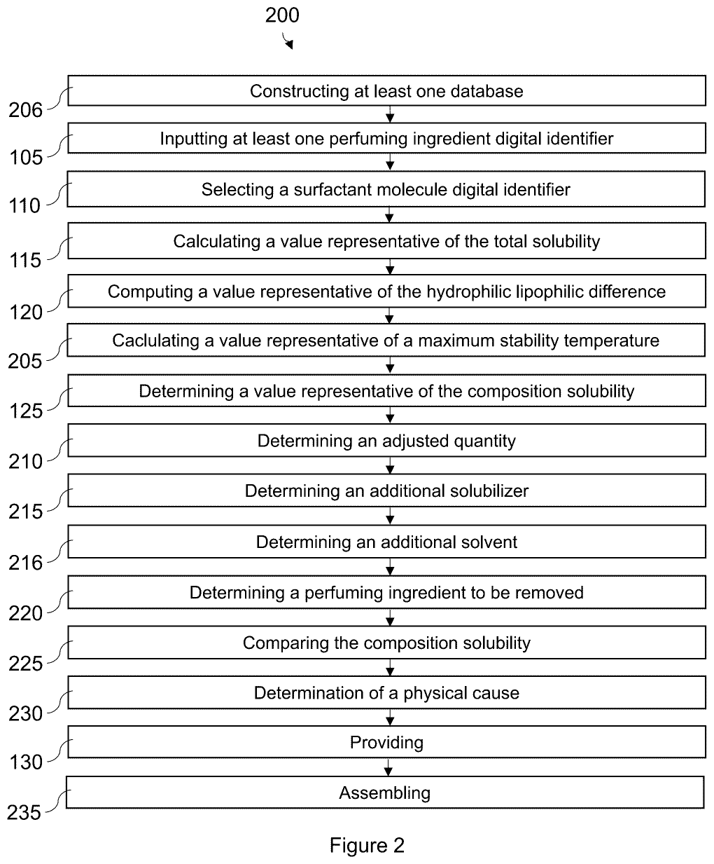

FIG. 2 shows additional embodiments and variants of the method 200 object of the present invention.

In particular embodiments, the method 200 object of the present invention comprises a step 205 of calculation, for the composition, by a computing system, of a value representative of a maximum stability temperature, said temperature being used during the step 125 of determining.

This step of calculation 205 is performed, for example, by a computer software executed by a computing system, such as shown in FIG. 7. During this step of calculation 205, any known equation may be used. For example, the maximum stability temperature may be calculated with the following formula:

$$T = \frac{H L D - C c + k \cdot EACN - ln ( S ) + f ( A ) - t \cdot 25}{- t}$$

where:
HLD is the hydrophilic lipophilic difference of said mixture,
Cc is the characteristic curvature of the surfactant molecule,
EACN is the equivalent alkane carbon number of the perfuming ingredient,
S is the concentration of electrolyte in the mixture,
A is the concentration of alcohol in the mixture,
k, f, and t are constants.

Such a value may be required as some perfuming ingredients in a composition might decrease the cloud point of the surfactant base and become insoluble. This additional parameter can be added to consider the temperature phase boundary of the microemulsion. The calculated value may be combined with the total solubility calculated and of a hydrophilic lipophilic difference computed to form a set of coordinates in a three-dimensional space or mathematically combined to produce a single value.

In particular embodiments, the method 200 object of the present invention comprises which comprises a step 210 of determining, by a computing system, an adjusted quantity of at least one input physical perfuming ingredient represented by at least one input physical perfuming ingredient digital identifier as a function of the determined composition solubility in the aqueous environment to reach a target hydrophilic lipophilic difference, the step 130 of providing being configured to provide the determined adjusted concentration.

This step of determining 210 an adjusted quantity is performed, for example, by a computer software executed by a computing system, such as shown in FIG. 7. During this step of determining 210, either the equation used in the step of calculating 115 and/or the equation used in the step of computing 120 is used so that the total solubility and/or the hydrophilic lipophilic difference reach a determined value representative of an applicability domain. This can be achieved by adjusting the total fragrance oil concentration or single fragrance ingredients.

The values representative of the applicability domain are either predetermined or defined by a user prior to the step of determining 210, during a step (not represented) of defining an applicability domain upon a computer interface.

The applicability domain depends on the content of surfactant, electrolytes, and solvents, and is therefore specific for each application base. The domain can be mapped out using a series of test fragrance mixtures with a variety of EACN values. Maximum solubilization concentrations are evaluated experimentally and plotted as a function of the HLD of the mixture. Alternatively, the solubilization concentrations are calculated based on the equation above.

Maximum solubilization concentrations can be experimentally determined, for example by adding excess amounts of the perfuming ingredient to a water phase containing surfactant micelles, followed by stirring for 24 h in a temperature-controlled environment. After separating the two phases, excess fragrance oil and aqueous phase, respectively, the quantity of the solubilized perfuming ingredient in the aqueous phase can be analytically determined, e.g., by GC/MS, GC/FID or by UV/Vis spectroscopy.

Alternatively, a series of samples of water phase containing surfactant micelles is prepared and increasing amounts of a perfuming ingredient are added to each of the samples. After stirring for 24 h in a temperature-controlled environment and further incubation of several hours up to several days the samples are visually assessed, and the turbidity is determined using a turbidimeter. The highest concentration of the perfuming ingredient, which yields a clear and transparent sample, in the absence of phase separation, represents the solubility limit of the perfuming ingredient.

The term transparent means that the aqueous phase in the absence of coloring or fluorescent agents have transmittance values in the visible light (500-800 nm) of 100% at a path length of 1 cm referenced against demineralized water.

In particular embodiments, the method 200 object of the present invention comprises a step 215 of determining, by a computing system, at least one additional physical solubilizer digital identifier, representative of a physical solubilizer, to be input in the composition as a function of the determined composition solubility in the aqueous environment to reach a target hydrophilic lipophilic difference, the step 130 of providing being configured to provide the determined additional solubilizer digital identifier.

This step of determining 215 at least one additional solubilizer digital identifier to be input is performed, for example, by a computer software executed by a computing system, such as shown in FIG. 7. During this step of determining 215, an equation linking hydrophilic lipophilic difference and characteristic curvature, such as the one disclosed above, is used to determine a target characteristic curvature as a function of a target hydrophilic lipophilic difference corresponding to an applicability domain that is either determined or set.

The values representative of the applicability domain are either predetermined or defined by a user prior to the step of determining 215, during a step (not represented) of defining an applicability domain upon a computer interface.

The target characteristic curvature may then be obtained from the current characteristic curvature by adding solubilizers to the composition to be materialized, each solubilizer being associated to a characteristic curvature. A solubilizer digital identifier is selected as a function of the characteristic curvature associated to said solubilizer and the quantity of said solubilizer to be added to the composition to reach the target hydrophilic lipophilic difference.

In variants, the step of determining 215 is configured to provide an adjusted quantity of solubilizer to be added to the composition.

In particular embodiments, the method 200 object of the present invention comprises a step 216 of determining, by a computing system, at least one additional physical solvent digital identifier, representative of a physical solvent, to be input in the composition as a function of the determined composition solubility in the aqueous environment to reach a target hydrophilic lipophilic difference, the step 130 of providing being configured to provide the determined additional solvent digital identifier.

This step of determining 216 at least one additional solvent digital identifier to be input is performed, for example, by a computer software executed by a computing system, such as shown in FIG. 7. During this step of determining 216, an equation linking hydrophilic lipophilic difference and characteristic curvature, such as the one disclosed above, is used to determine a target characteristic curvature as a function of a target hydrophilic lipophilic difference corresponding to an applicability domain that is either determined or set.

The values representative of the applicability domain are either predetermined or defined by a user prior to the step of determining 216, during a step (not represented) of defining an applicability domain upon a computer interface.

The target characteristic curvature may then be obtained from the current characteristic curvature by adding solubilizers to the composition to be materialized, each solvent being associated to a characteristic curvature. A solvent digital identifier is selected as a function of the characteristic curvature associated to said solvent and the quantity of said solvent to be added to the composition to reach the target hydrophilic lipophilic difference.

In variants, the step of determining 216 is configured to provide an adjusted quantity of solvent to be added to the composition.

In particular embodiments, the method 200 object of the present invention comprises a step 220 of determining, by a computing system, at least one physical perfuming ingredient digital identifier to be removed as a function of the determined composition solubility in the aqueous environment, the step 130 of providing being configured to provide the determined corresponding physical perfuming ingredient digital identifier.

The step of determining 220 at least one physical perfuming ingredient digital identifier to be removed is performed, for example, by a computer software executed by a computing system, such as shown in FIG. 7. This step of determining 220 functions similarly to the step of determining 210 an adjusted quantity where the quantity is reduced to zero.

In particular embodiments, the method 200 object of the present invention comprises:
a step 225 of comparing, by a computing system, the determined composition solubility in an aqueous environment to at least one threshold value and
a step 230 of determination, by a computing system, of at least one physical cause digital identifier for the result of the step of comparing,
   the step 130 of providing being configured to provide each said physical cause digital identifier.

The step of comparing 225 is performed, for example, by a computer software executed by a computing system. During this step of comparing 225, the aggregated value or the values of coordinates of the solubility in an aqueous environment is or are compared to at least one threshold value representative of an applicability domain. Depending on the outcome of the step of comparing 225, a physical cause may be determined.

The terms “physical cause” refer to, in this instance, the reasons why the materialized composition would fail to meet the applicability domain criteria that are set for the intended use-case of the composition.

Such causes might be, for example:
the dosage of the fragrance composition is too high,
the dosage of a particular perfuming ingredient is too high,
the fragrance composition used is too apolar or too polar and/or
the surfactant is too apolar or too polar.

Each cause might be represented, in a database, with a particular digital identifier.

The step of determination 230 is performed, for example, by a computer software executed by a computing system. During this step of determination 230, the result of the step of comparing 225 is compared, for example, to specific difference thresholds, each threshold being representative of a particular physical cause.

At least one determined physical cause may be shown during the step of providing 130.

In particular embodiments, the method 200 object of the present invention comprises a step 235 of assembling the composition of perfuming ingredients represented by each input physical perfuming ingredient digital identifier.

The step 235 of assembling is performed by any means to assemble a composition known to the field of perfumery. Such a step of assembling 235 may involve a manufacturing facility for example. During this step of assembling 235, each input perfuming ingredient and each solubilizer is assembled so as to reflect the quantities of each said perfuming ingredient and solubilizer obtained using the method, 100 or 200, object of the present invention.

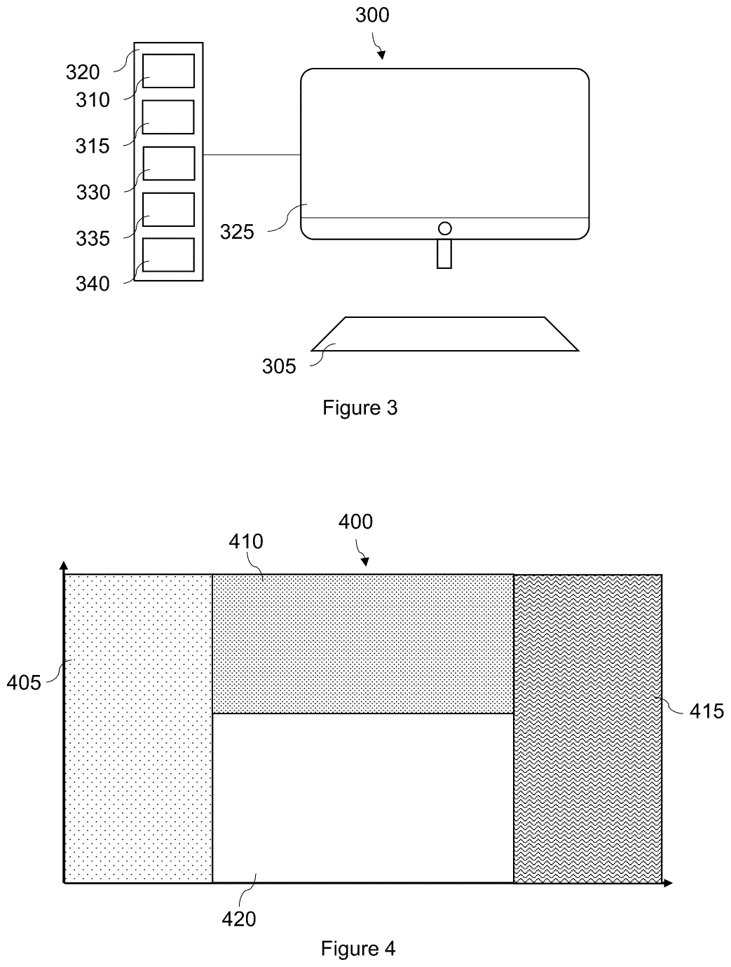

FIG. 3 represents, schematically, a particular embodiment of the method 300 object of the present invention. This composition solubility in an aqueous environment comprising a surfactant molecule determination system 300, comprises:
means 310 of inputting, upon a computer interface 305, at least one physical perfuming ingredient digital identifier, said physical perfuming ingredient digital identifier being representative of a physical perfuming ingredient, the resulting input being representative of a composition of the represented physical perfuming ingredients,
means 315 of selecting, upon a computer interface, at least one physical surfactant molecule digital identifier, said surfactant molecule digital identifier being representative of a physical surfactant molecule,
means 330 of calculating, by a computing system 320, for at least one input physical perfuming ingredient digital identifier, a value representative of the total solubility of the corresponding physical perfuming ingredient in an aqueous environment comprising micelles of at least one physical surfactant molecule corresponding to at least one selected physical surfactant molecule digital identifier, wherein the aqueous environment, at least one said selected physical surfactant, and the composition define a mixture,
means 335 of computing, by a computing system, for at least one input physical perfuming ingredient digital identifier in the composition and for at least one selected physical surfactant molecule digital identifier in the mixture, a value representative of the hydrophilic lipophilic difference of said mixture,
means 340 of determining, by a computing system, for at least one input physical perfuming ingredient digital identifier, a value representative of the composition solubility in the aqueous environment, as a function of a total solubility calculated and of a hydrophilic lipophilic difference computed, and
means 325 of providing, upon a computer interface, at least one determined composition solubility.

Examples of the means to be used are described in relation to the methods, 100 and 200, object of the present invention disclosed in regard to FIGS. 1 and 2.

For example, the means 310 of inputting may correspond to a computer program ran upon a computing system 320 or to a controller, associated with a keyboard 305 and/or mouse and/or touchscreen.

The means of selecting 315 may be similar to the means 310 of inputting. The means of calculating 330, computing 335 and determining 340 may correspond to dedicated or integrated instructions to be executed in the form of a computer software ran upon the computing system 320.

The means of providing 325 may correspond to a computer program ran upon the computing system 320 in association with a computer screen 325 or to a controller of said computer screen 325.

FIG. 4 shows a two-dimensional graph 400 that represents the composition solubility in an aqueous environment, the y-axis representing the maximum solubility of the composition and the x-axis representing the hydrophilic lipophilic difference of the composition.

In this figure, four different domains are identified:
a first region 405 corresponds to unviable compositions due to the fact that the surfactant is too polar, or the oil is too apolar,
a second region 410 corresponds to unviable compositions due to the fact that the dosage, in terms of concentration, of the composition or of a single ingredient is too high,
a third region 415 corresponds to unviable compositions due to the fact that the surfactant is too apolar or the oil is too polar and
a fourth region 420 corresponds to viable compositions.

Maximum solubility limit and hydrophilic lipophilic difference are calculated for a composition and positioned onto a two-dimensional graph 405. If the composition is positioned in the any one of the first, second or third regions, 410, 415 or 420, the composition is insoluble, whereas the fourth region 420 means the composition is soluble. The extent of the fourth region 420 depends on surfactant concentration (vertical) and surfactant type and composition of the mixture, respectively (horizontal). Depending on where the fragrance is situated inside the first, second or third regions, 410, 415 or 420, it is possible to determine the underlying cause: too high dosage, or fragrance—surfactant polarity mismatch.

Subsequently, formulation guidelines can be established to improve the solubility of a composition. Addition of solvents and solubilizers to the fragrance can shift its position and direct it towards the fourth region 420 and make the composition more soluble.

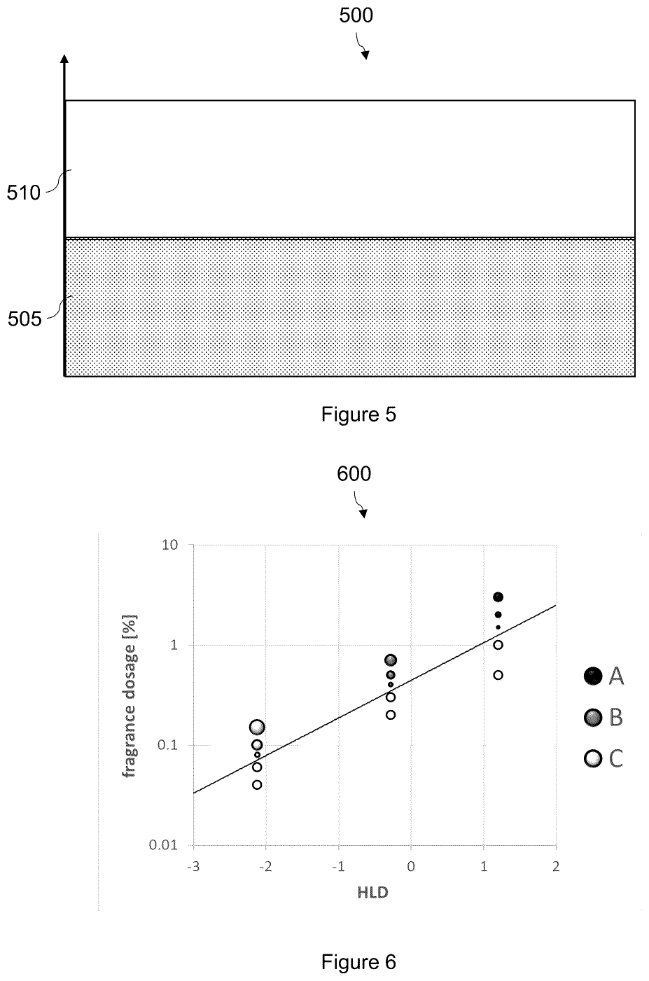

FIG. 5 shows a two-dimensional graph 505 that represents the maximum stability temperature of a composition. Temperature limit and matching factor are calculated for a composition and positioned onto a two-dimensional graph 505. If the composition is positioned in a first region 505 it's insoluble and within a second region 510 it is soluble. Typically, by adding too much of amphiphilic compositions it may become insoluble by shifting the phase transition (cloud point) into the lower/ambient temperature region. By the addition of solubilizers the cloud point can be increased so that the composition becomes soluble at a given temperature.

FIG. 6 shows the values 600 for the calculated insoluble fraction of three different fragrance compositions (A, B and C) at a given dosage in the surfactant base (y-axis). At lower dosages the insoluble fraction is zero (empty symbols). At higher dosages the insoluble fraction becomes greater than zero and increases with the added amount of the fragrance composition to the surfactant base (displayed by the relative size of the filled symbols). The three fragrance compositions are depicted at their calculated HLD values in the mixture. The solid line represents the solubility limit as a function of the HLD of the mixture, where the domain below the line represents the region 420 corresponding to viable compositions.

FIG. 7 represents a block diagram that illustrates an example computer system 700 with which may implement an embodiment of the present invention. Such a computer system 700 is also referred to as a computing system or a computing device in the present document. In the example of FIG. 7, a computer system 705 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

The computer system 705 includes an input/output (10) subsystem 720 which may include a bus and/or other communication mechanism(s) for communicating information and/or instructions between the components of the computer system 705 over electronic signal paths. The I/O subsystem 720 may include an I/O controller, a memory controller and at least one I/O port. The electronic signal paths are represented schematically in the drawings, for example as lines, unidirectional arrows, or bidirectional arrows.

At least one hardware processor 710 is coupled to the I/O subsystem 720 for processing information and instructions. Hardware processor 710 may include, for example, a general-purpose microprocessor or microcontroller and/or a special-purpose microprocessor such as an embedded system or a graphics processing unit (GPU) or a digital signal processor or ARM processor. Processor 710 may comprise an integrated arithmetic logic unit (ALU) or may be coupled to a separate ALU.

Computer system 705 includes one or more units of memory 725, such as a main memory, which is coupled to I/O subsystem 720 for electronically digitally storing data and instructions to be executed by processor 710. Memory 725 may include volatile memory such as various forms of random-access memory (RAM) or other dynamic storage device. Memory 725 also may be used for storing temporary variables or other intermediate information during execution of instructions to be executed by processor 710. Such instructions, when stored in non-transitory computer-readable storage media accessible to processor 710, can render computer system 705 into a special-purpose machine that is customized to perform the operations specified in the instructions.

Computer system 705 further includes non-volatile memory such as read only memory (ROM) 730 or other static storage device coupled to the I/O subsystem 720 for storing information and instructions for processor 710. The ROM 730 may include various forms of programmable ROM (PROM) such as erasable PROM (EPROM) or electrically erasable PROM (EEPROM). A unit of persistent storage 715 may include various forms of non-volatile RAM (NVRAM), such as FLASH memory, or solid-state storage, magnetic disk, or optical disk such as CD-ROM or DVD-ROM and may be coupled to I/O subsystem 720 for storing information and instructions. Storage 715 is an example of a non-transitory computer-readable medium that may be used to store instructions and data which when executed by the processor 710 cause performing computer-implemented methods to execute the techniques herein.

The instructions in memory 725, ROM 730 or storage 715 may comprise one or more sets of instructions that are organized as modules, methods, objects, functions, routines, or calls. The instructions may be organized as one or more computer programs, operating system services, or application programs including mobile apps.

The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 705 may be coupled via I/O subsystem 720 to at least one output device 735. In one embodiment, output device 735 is a digital computer display. Examples of a display that may be used in various embodiments include a touch screen display or a light-emitting diode (LED) display or a liquid crystal display (LCD) or an e-paper display. Computer system 705 may include other type(s) of output devices 735, alternatively or in addition to a display device. Examples of other output devices 735 include printers, ticket printers, plotters, projectors, sound cards or video cards, speakers, buzzers or piezoelectric devices or other audible devices, lamps or LED or LCD indicators, haptic devices, actuators, or servos.

At least one input device 740 is coupled to I/O subsystem 720 for communicating signals, data, command selections or gestures to processor 710. Examples of input devices 740 include touch screens, microphones, still and video digital cameras, alphanumeric and other keys, keypads, keyboards, graphics tablets, image scanners, joysticks, clocks, switches, buttons, dials, slides.

Another type of input device is a control device 745, which may perform cursor control or other automated control functions such as navigation in a graphical interface on a display screen, alternatively or in addition to input functions. Control device 745 may be a touchpad, a mouse, a trackball, or cursor direction keys for communicating direction information and command selections to processor 710 and for controlling cursor movement on display 735. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 740 may include a combination of multiple different input devices, such as a video camera and a depth sensor.

In another embodiment, computer system 705 may comprise an internet of things (loT) device in which one or more of the output device 735, input device 740, and control device 745 are omitted. Or, in such an embodiment, the input device 740 may comprise one or more cameras, motion detectors, thermometers, microphones, seismic detectors, other sensors or detectors, measurement devices or encoders and the output device 735 may comprise a special-purpose display such as a single-line LED or LCD display, one or more indicators, a display panel, a meter, a valve, a solenoid, an actuator or a servo.

Computer system 705 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 705 in response to processor 710 executing at least one sequence of at least one instruction contained in main memory 725. Such instructions may be read into main memory 725 from another storage medium, such as storage 715. Execution of the sequences of instructions contained in main memory 725 causes processor 710 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may comprise non-volatile media and/or volatile media. Non-volatile media includes, for example, optical or magnetic disks, such as storage 715. Volatile media includes dynamic memory, such as memory 725. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 720. Transmission media can also take the form of acoustic or light waves, such as those generated during radio-wave and infra-red data communications.

Various forms of media may be involved in carrying at least one sequence of at least one instruction to processor 710 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 705 can receive the data on the communication link and convert the data to a format that can be read by computer system 705. For instance, a receiver such as a radio frequency antenna or an infrared detector can receive the data carried in a wireless or optical signal and appropriate circuitry can provide the data to I/O subsystem 720 such as place the data on a bus. I/O subsystem 720 carries the data to memory 725, from which processor 710 retrieves and executes the instructions. The instructions received by memory 725 may optionally be stored on storage 715 either before or after execution by processor 710.

Computer system 705 also includes a communication interface 760 coupled to bus 720. Communication interface 760 provides a two-way data communication coupling to network link(s) 765 that are directly or indirectly connected to at least one communication networks, such as a network 770 or a public or private cloud on the Internet. For example, communication interface 760 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 770 broadly represents a local area network (LAN), wide-area network (WAN), campus network, internetwork, or any combination thereof. Communication interface 760 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 760 sends and receives electrical, electromagnetic, or optical signals over signal paths that carry digital data streams representing various types of information.

Network link 765 typically provides electrical, electromagnetic, or optical data communication directly or through at least one network to other data devices, using, for example, satellite, cellular, Wi-Fi, or BLUETOOTH technology. For example, network link 765 may provide a connection through a network 770 to a host computer 750.

Furthermore, network link 765 may provide a connection through network 770 or to other computing devices via internetworking devices and/or computers that are operated by an Internet Service Provider (ISP) 775. ISP 775 provides data communication services through a world-wide packet data communication network represented as internet 780. A server computer 755 may be coupled to internet 780. Server 755 broadly represents any computer, data center, virtual machine, or virtual computing instance with or without a hypervisor, or computer executing a containerized program system such as DOCKER or KUBERNETES. Server 755 may represent an electronic digital service that is implemented using more than one computer or instance and that is accessed and used by transmitting web services requests, uniform resource locator (URL) strings with parameters in HTTP payloads, API calls, app services calls, or other service calls. Computer system 705 and server 755 may form elements of a distributed computing system that includes other computers, a processing cluster, server farm or other organization of computers that cooperate to perform tasks or execute applications or services. Server 755 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 755 may comprise a web application server that hosts a presentation layer, application layer and data storage layer such as a relational database system using structured query language (SQL) or no SQL, an object store, a graph database, a flat file system or other data storage.

Computer system 705 can send messages and receive data and instructions, including program code, through the network(s), network link 765 and communication interface 760. In the Internet example, a server 755 might transmit a requested code for an application program through Internet 780, ISP 775, local network 770 and communication interface 760. The received code may be executed by processor 710 as it is received, and/or stored in storage 715, or other non-volatile storage for later execution.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 710. While each processor 710 or core of the processor executes a single task at a time, computer system 705 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

1. Composition solubility in an aqueous environment comprising a surfactant molecule determination method, comprising:
a step of inputting, upon a computer interface, at least one physical perfuming ingredient digital identifier, said physical perfuming ingredient digital identifier being representative of a physical perfuming ingredient, the resulting input being representative of a composition of the represented physical perfuming ingredients,
a step of selecting, upon a computer interface, at least one physical surfactant molecule digital identifier, said surfactant molecule digital identifier being representative of a physical surfactant molecule,
a step of calculating, by a computing system, for at least one input physical perfuming ingredient digital identifier, a value representative of the total solubility of the corresponding physical perfuming ingredient in an aqueous environment comprising micelles of at least one physical surfactant molecule corresponding to at least one selected physical surfactant molecule digital identifier, wherein the aqueous environment, at least one said selected physical surfactant, and the composition define a mixture,
a step of computing, by a computing system, for at least one input physical perfuming ingredient digital identifier in the composition and for at least one selected physical surfactant molecule digital identifier in the mixture, a value representative of the hydrophilic lipophilic difference of said mixture,
a step of determining, by a computing system, for at least one input physical perfuming ingredient digital identifier, a value representative of the composition solubility in the aqueous environment, as a function of a total solubility calculated and of a hydrophilic lipophilic difference computed, and
a step of providing, upon a computer interface, at least one determined composition solubility.

2. Method according to claim 1, which further comprises a step of calculation, for the mixture, by a computing system, of a value representative of a maximum stability temperature, said value being used during the step of determining a value representative of the composition solubility in an aqueous environment.

3. Method according to claim 2, in which the maximum stability temperature is calculated using the formula:
$$T = \frac{H L D - C c + k \cdot EACN - ln ( S ) + f ( A ) - t \cdot 25}{- t}$$
where:
HLD is the hydrophilic lipophilic difference of said mixture,
Cc is the characteristic curvature of the surfactant molecule,
EACN is the equivalent alkane carbon number of the perfuming ingredient,
S is the concentration of electrolyte in the mixture,
A is the concentration of alcohol in the mixture,
k, f, and t are constants.

4. Method according to claim 1, in which the total solubility for a perfuming ingredient is calculated using the formula:
Sₜₒₜ^{F}=S_{W}^{F}·(1+P_{O/W}^{F}·AF·V̅_{S}·c_{S}/(1−V̅_{S}·c_{S}))
where:
S_{W}^{F} is the perfuming ingredient solubility in water,
c_{S} is the concentration of micellized surfactant in the mixture,
V̅_{S} is the molecular volume of the surfactant molecule,
P_{O/W}^{F} is the n-octanol-water partition coefficient of the perfuming ingredient and
AF is the affinity factor of the surfactant molecule.

5. Method according to claim 1, in which the hydrophilic lipophilic difference for a perfuming ingredient is calculated using the formula:
HLD=ln(S)−k·EACN−f(A)+Cc−t·(ΔT)
where:
S is the concentration of electrolyte in the mixture,
EACN is the equivalent alkane carbon number of the perfuming ingredient,
A is the concentration of alcohol in the mixture,
ΔT is the temperature difference to a reference temperature of the mixture,
Cc is the characteristic curvature of the surfactant molecule,
k, f, and t are constants.

6. Method according to claim 1, which comprises a step of determining, by a computing system, an adjusted quantity of at least one input physical perfuming ingredient represented by at least one input physical perfuming ingredient digital identifier as a function of the determined composition solubility in an aqueous environment to reach a target hydrophilic lipophilic difference, the step of providing being configured to provide the determined adjusted concentration.

7. Method according to claim 1, which comprises a step of determining, by a computing system, at least one additional physical solubilizer digital identifier, representative of a physical solubilizer, to be input in the composition as a function of the determined composition solubility in the aqueous environment to reach a target hydrophilic lipophilic difference, the step of providing being configured to provide the determined additional solubilizer digital identifier.

8. Method according to claim 1, which comprises a step of determining, by a computing system, at least one additional physical solvent digital identifier, representative of a physical solvent, to be input in the composition as a function of the determined composition solubility in the aqueous environment to reach a target hydrophilic lipophilic difference, the step of providing being configured to provide the determined additional solvent digital identifier.

9. Method according to claim 1, which comprises a step of determining, by a computing system, at least one physical perfuming ingredient digital identifier to be removed as a function of the determined composition solubility in the aqueous environment to reach a target hydrophilic lipophilic difference, the step of providing being configured to provide the determined corresponding physical perfuming ingredient digital identifier.

10. Method according to claim 1, which comprises:
a step of comparing, by a computing system, the determined composition solubility in the aqueous environment to at least one threshold value and
a step of determination, by a computing system, of at least one physical cause digital identifier for the result of the step of comparing,
the step of providing being configured to provide each said physical cause digital identifier.

11. Method according to claim 1, which comprises a step of assembling the composition.

12. Method according to claim 1, which comprises a step of constructing at least one database associating:
physical perfuming ingredient digital identifiers to at least one physical perfuming ingredient parameter value, and
physical surfactant molecule digital identifiers to at least one physical surfactant molecule parameter value,
wherein at least one said physical perfuming ingredient parameter value and at least one physical surfactant molecule parameter value are used during the step of calculating and/or the step of computing.

13. Composition solubility in an aqueous environment comprising a surfactant molecule determination system, comprising:
means of inputting, upon a computer interface, at least one physical perfuming ingredient digital identifier, said physical perfuming ingredient digital identifier being representative of a physical perfuming ingredient, the resulting input being representative of a composition of the represented physical perfuming ingredients,
means of selecting, upon a computer interface, at least one physical surfactant molecule digital identifier, said surfactant molecule digital identifier being representative of a physical surfactant molecule,
means of calculating, by a computing system, for at least one input physical perfuming ingredient digital identifier, a value representative of the total solubility of the corresponding physical perfuming ingredient in an aqueous environment comprising micelles of at least one physical surfactant molecule corresponding to at least one selected physical surfactant molecule digital identifier, wherein the aqueous environment, at least one said selected physical surfactant, and the composition define a mixture,
means of computing, by a computing system, for at least one input physical perfuming ingredient digital identifier in the composition and for at least one selected physical surfactant molecule digital identifier in the mixture, a value representative of the hydrophilic lipophilic difference of said mixture,
means of determining, by a computing system, for at least one input physical perfuming ingredient digital identifier, a value representative of the composition solubility in the aqueous environment, as a function of a total solubility calculated and of a hydrophilic lipophilic difference computed, and
means of providing, upon a computer interface, at least one determined composition solubility.
